(19) United States
(12) Patent Application Publication
(10) Pub. No.: US 2011/0208145 A1
(43) Pub. Date: Aug. 25, 2011

(54) Fluid Management System

(76) Inventors: Le Zhang, Lutherville, MD (US); Yunzhang Wang, Duncan, SC (US); Carnevale Sally, Greenville, SC (US); Robert M. MacMeccan, Greer, SC (US); Jeeyoung Choi, Santa Clara, CA (US); Keith A. Keller, Spartanburg, SC (US); Patrick R. Carroll, Spartanburg, SC (US); James M. Spry, Inman, SC (US); Robert Champion, Spartanburg, SC (US)

(21) Appl. No.: 13/019,332

(22) Filed: Feb. 2, 2011

Related U.S. Application Data
(60) Provisional application No. 61/306,726, filed on Feb. 22, 2010.
(60) Provisional application No. 61/330,598, filed on May 3, 2010.
(60) Provisional application No. 61/406,631, filed on Oct. 26, 2010.

(51) Int. Cl. A61F 13/51; A61L 15/22; A61L 15/28

(52) U.S. Cl. 604/368; 604/378; 604/369; 604/372; 604/374

(57) ABSTRACT

A fluid management cover system of launderable and durable character including at least one fluid transport layer with at least one absorptive reservoir layer disposed in underlying relation to the fluid transport layer. The upper surface of the absorptive reservoir layer is characterized by a greater capillarity than the adjacent surface of the fluid transport layer such that fluid is drawn away from the fluid transport layer and is held within the absorptive reservoir layer. At least one pressure distribution layer incorporating material that at least partially recovers following compression is disposed at a position below the absorptive reservoir layer. An optional skin contacting layer may be disposed above the fluid transport layer and an optional backing layer may be disposed in underlying relation to other layers.

### RELATED APPLICATIONS

This application claims priority to provisional applications 61/306,726 filed Feb. 22, 2010, 61/330,598 filed May 3, 2010, and 61/406,631 filed Oct. 26, 2010, all of which are herein incorporated by reference in their entirety.

### TECHNICAL FIELD

The present invention relates generally to a fluid transport system, and more particularly, to a system for management of fluid transport away from a user, for example on a mattress or seated in a chair including a wheelchair or the like. More particularly, the invention relates to a system incorporating a composite having layers of varying capillarity at their surfaces to promote the controlled withdrawal of fluid away from a user in combination with pressure redistribution and recovery following compression.

### BACKGROUND

During the treatment of various injuries and illnesses, it may be necessary for a person to remain bedridden for an extended period of time. Persons with illness or injury may also be confined to a seated condition for long periods of time and may require the use of a wheelchair to move from place to place. Persons with wounds will normally have a bandage or other covering protecting those wounds to control the discharge of fluids in the form of wound exudates or the like. However, upon saturation of the covering bandage the wound fluid may be released to an underlying mattress, chair, or other underlying support surface. In addition to excretions from wounds, some patients may suffer from medical conditions which make it difficult to control urinary and bowel functions. In the event of urinary or bowel release, fluids may migrate to the underlying mattress, chair, or other underlying support surface.

As will be appreciated, in the event that bodily fluids reach the mattress, chair, or other underlying support surface, staining and contamination may occur. In that event, the mattress, chair, or other underlying support surface typically must undergo a rigorous cleaning procedure. In extreme cases, the mattress, chair, or other underlying support surface may have to be discarded. Due to the biological hazards that may be associated with discharged bodily fluids, such cleaning and/or discard procedures may be relatively complex and expensive to carry out.

In the past, pads have been used for disposition in covering relation to an underlying mattress, chair, or other underlying support surface for purposes of blocking the transmission of bodily fluids from the user to the underlying mattress, chair, or other underlying support surface. However, such pads have typically merely contained the fluid at the surface such that it may remain in contact with the user. Such contact with the expelled fluid is generally undesirable due to the potential for skin irritation, contamination of other wound sites, and the like. Another problem with prior pads has been a lack of resiliency in the thickness dimension to provide pressure redistribution during loading with recovery following loading. Thus, the pads may tend to exhibit a breakdown in thickness at zones normally corresponding to the position of the user. Such a breakdown in thickness may reduce the effectiveness of the pad in containing fluids at those locations.

A need therefore exists for a system that controls discharges of bodily fluids without subjecting a user to prolonged contact with such bodily fluids and which accommodates and recovers from compressive loading during use. In particular, a need exists for a system that moves expelled bodily fluids away from a user and holds such fluids remotely from the user without passage to an underlying mattress, chair, or other underlying support surface while concurrently accommodating compressive loading in a wet state.

### BRIEF SUMMARY

In accordance with one exemplary embodiment, the present invention provides advantages and alternatives over the prior art by providing a fluid management cover system of launderable and durable character including at least one fluid transport layer with at least one absorptive reservoir layer disposed in underlying relation to the fluid transport layer. The upper surface of the absorptive reservoir layer is characterized by a greater capillarity than the adjacent surface of the fluid transport layer such that fluid is drawn away from the fluid transport layer and is held within the absorptive reservoir layer. At least one pressure distribution layer incorporating material that at least partially recovers following compression is disposed at a position below the absorptive reservoir layer. An optional skin contacting layer may be disposed above the fluid transport layer and an optional backing layer may be disposed in underlying relation to other layers.

### BRIEF DESCRIPTION OF THE DRAWINGS

The accompanying drawings, which are incorporated in and which constitute a part of the specification, illustrate various potentially preferred embodiments of the present invention, and together with the generally description above and the detailed description below, serve to explain the principles of the invention wherein:

FIG. 1 is a perspective view of one embodiment of an exemplary mattress cover including a top panel and side panels which may be elastomeric or non-elastomeric;

FIG. 1A is a schematic view of a wheelchair including an exemplary chair cover pad;

FIG. 2 is a perspective sectional view taken generally along line 2-2 in FIG. 1 or through the chair cover pad of FIG. 1A illustrating an exemplary composite construction for the top panel of a mattress pad or chair cover pad with an optional overlying skin contact layer;

FIG. 3 is a view similar to FIG. 2 incorporating a dual function reservoir and pressure redistribution layer with absorptive elements blended intimately with pressure redistribution elements;

FIG. 4 is a view similar to FIG. 3 incorporating a dual function reservoir and pressure redistribution layer with a variable concentration of absorptive elements and pressure redistribution elements disposed in a gradient through the thickness dimension of the dual function layer.

FIG. 5 is a view similar to FIG. 2 illustrating a composite structure incorporating a dual function transport and reservoir layer providing combined transport and fluid reservoir functions disposed above a pressure redistribution layer;

FIG. 6 is a plan view of a laid-in fabric suitable for use as a dual function fluid transport and reservoir layer in the composite structure of FIG. 5;

FIG. 7 is a schematic view illustrating an exemplary construction for a portion of a mattress cover composite providing enhanced force redistribution and recovery characteristics;

FIG. 8 is a schematic view illustrating an exemplary pile fabric useful as a fluid transport layer and

FIG. 8A is a schematic view illustrating an exemplary pile fabric of FIG. 8A following face finishing treatment.

FIG. 9 is a perspective view of one embodiment of an exemplary mattress cover;

Before exemplary embodiments of the invention are explained in detail, it is to be understood that the invention is in no way limited in its application to the details of construction and/or to the arrangements of the components set forth in the following description or illustrated in the drawings. Rather, the invention is capable of other embodiments and of being practiced or carried out in various ways. Also, it is to be understood that the phraseology and terminology used herein are for purposes of description only and should not be regarded as limiting. The use herein of “including”, “comprising”, and variations thereof is meant to encompass the items listed thereafter and equivalents, as well as additional items and equivalents thereof.

### DETAILED DESCRIPTION

As utilized herein, the term “capillarity” refers to the ability of a material to retain or expel fluid. In this regard, the capillarity of a material is determined by the pore size of the material as well as the contact angle with the fluid in question. Capillarity is measured by the ability of a material to either resist pressure when submerged to a given depth in an aqueous fluid or to draw aqueous fluid to a given height. Thus, the capillarity is the height of fluid drawn up (if positive) or height immersed (if negative) times the fluid density. The capillarity is defined by the following equation:

$$C = \frac{2 γ cos θ}{g r}$$

γ is the liquid-air surface tension (energy/area)

θ is the contact angle

g is acceleration due to gravity (length/time²)

r is pore radius

In this regard, the effective pore radius for a fabric with multiple pore sizes is generally dependent upon the character of the fabric. For materials that draw fluid inwardly, the effective radius tends to be dominated by the smaller pore sizes. For materials that expel fluid, the effective radius tends to be dominated by the larger pore sizes. As will be recognized by those of skill in the art, capillarity levels may be measured directly by using known techniques including wetting tests such as wicking and dewetting tests such as liquid extrusion porosimetry or porometry. Fluid includes moisture as well as other fluids such as sweat and urine.

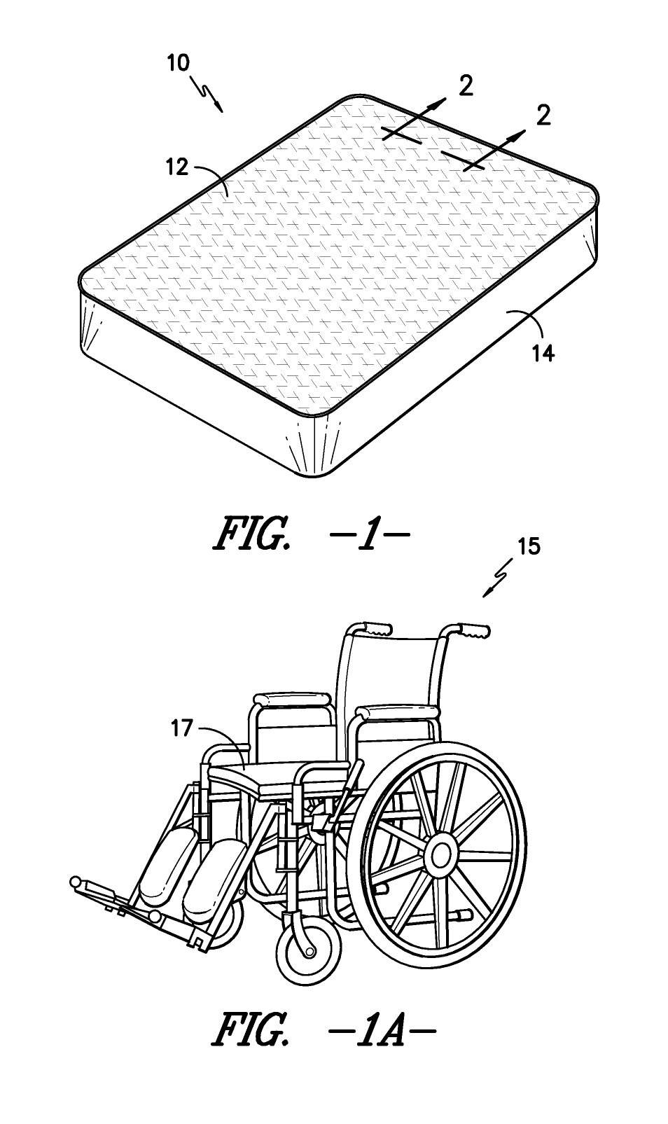

Reference will now be made to the drawings wherein to the extent possible, like elements are designated by like reference numerals throughout the various views. FIG. 1 illustrates an exemplary mattress cover 10 including a top panel 12 and a skirt 14. In the illustrated embodiment, the top panel 12 has a quilted construction including an arrangement of quilting stitches forming stitch lines across the surface of the top panel 12. In this regard, the stitch lines are preferably spaced at increments of about 1 inch to about 12 inches, and more preferably, about 2 inches to about 8 inches and most preferably about 2 inches to about 5 inches relative to one another. The system is preferably static, meaning that no motors or fans are involved in its operation. This system is advantaged as it can cover the entire surface of the object to be protected and lie underneath the patient's entire body providing a full body moisture management system.

As will be appreciated, the mattress cover 10 may be disposed in fitted relation about an underlying mattress (not shown) and may then be periodically removed for laundering. If desired, the skirt 14 may have elastomeric character so as to facilitate a fitted relation about the mattress. However, the skirt may also be substantially inelastic or include only localized elastomeric elements such as an elastic band or the like around the bottom. Alternatively, it is contemplated that the mattress cover 10 may be devoid of a skirt. In such an embodiment, the top panel 12 may simply be placed on top of the underlying mattress. Alternatively, it is contemplated that the skirt 14 may be only a partial skirt of elastic or inelastic character. In any embodiment that may be used, the mattress cover 10 may also include handles to facilitate moving people with the mattress cover 10 acting as a sliding or support surface.

FIG. 1A illustrates an exemplary seating structure 15 in the form of a wheelchair for support of a user (not shown). A seating pad 17 formed from a multi-layer composite structure as will be described further hereinafter is disposed in at least partial covering relation to a support surface of the seating structure 15. If desired, the seating pad 17 may include a skirt of elastomeric character so as to facilitate a fitted relation about the support surface of the chair. If used, a skirt may also be substantially inelastic or include only localized elastomeric elements such as an elastic band or the like around the bottom. Alternatively, it is contemplated that the seating pad 17 may be devoid of a skirt. In such an embodiment, seating pad 17 may simply be placed on top of the underlying support surface.

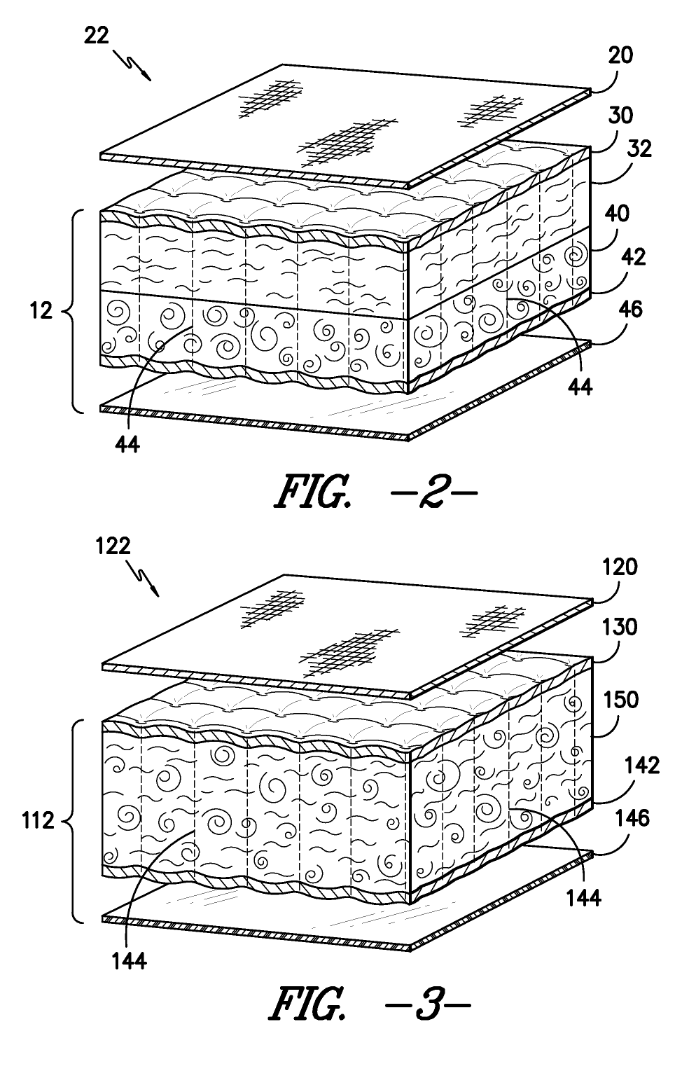

Regardless of whether or not the mattress cover 10 or seating pad 17 incorporates a skirt, such structures define a protective barrier interposed between a user and the underlying structure. FIG. 2 illustrates a first exemplary embodiment for a composite structure forming the top panel 12 of an exemplary mattress cover 10 or seating pad 17. In FIG. 2 the composite structure forming the top panel 12 or seating pad 17 is illustrated in combination with an optional overlying skin contact layer 20 to define a cooperating fluid management system 22 which is adapted to absorb and retain bodily fluids such as blood, sweat, wound exudates, urine, and the like as may be emitted by a user. In this regard, it is to be understood that the seating pad 17 may have the same layered construction as the top panel 12. Thus, all discussion and description relating to features of the top panel 12 is likewise applicable to the seating pad 17. Preferably, the fluid management system is launderable. In one embodiment, the system may be laundering in an industrial laundering more than 5 times, 20 times, 35 times, or 50 times without significant deterioration of its functionalities.

As illustrated, the exemplary fluid management system 22 includes an unattached skin contact layer 20 which may be in virtually any form useful in contacting a user's skin surface. The skin contacting layer 20 is typically of a form which is removed and replaced on a relatively high frequency basis. That is, the skin contacting layer 20 typically is in place for a relatively short duration and is removed and replaced much more frequently than the underlying mattress cover. By way of example only, and not limitation, the skin contacting layer 20 may be in the form of a bandage, a sheet, a gown, a diaper or virtually any other generally permeable structure. Materials of construction for the skin-contacting layer 20 may include knit, woven or non-woven textiles, apertured films, or the like.

It is to be understood that the skin-contacting layer 20 is purely optional. However, the presence of such a skin-contacting layer 20 may be desirable to establish an initial barrier layer which can act to collect small volumes of expelled fluids such as from a diaper without involving the underlying mattress cover 10 or seating pad 17. Since the skin-contacting layer 20 can be removed easily, the user will not experience prolonged exposure to the expelled fluids and will avoid any serious discomfort.

To deal with enhanced levels of discharge that are not contained by the skin contacting layer 20, the exemplary top panel 12 of the mattress cover 10 or seating pad 17 incorporates a multi-layer structure with different capillarity levels at different positions through the thickness dimension. Throughout this disclosure, it should be understood that by the term “layer” is meant any single or multi-level structure which occupies a substantially contiguous zone through the height dimension of the top panel 12. Thus, it is to be understood that any of the structures identified as “layers” within the present invention may be formed from a single ply, multiple plys, or arrangement of discreet particle elements held within the defined zone.

As depicted in FIG. 2, in a first embodiment the top panel 12 or seating pad 17 is a multi-layered composite having a fluid transport layer 30 positioned in overlying relation to an absorptive reservoir layer 32. The fluid transport layer 30 includes an upper first surface having a first capillarity and a lower second surface having a second capillarity. The first capillarity and the second capillarity may be either the same or different depending upon the construction of the fluid transport layer 30.

The absorptive reservoir layer 32 has an upper first surface and a lower second surface and is positioned such that its upper first surface is adjacent to the lower second surface of the fluid transfer layer 30. The capillarity of the upper surface of the absorptive reservoir layer 32 is greater than the capillarity of the adjacent lower surface of the fluid transfer layer 30. Thus, the absorptive reservoir layer 32 is a reservoir for the fluids taken out by the fluid transport layer 30.

The fluid transport layer 30 of the fluid management system 22 may be any suitable material that is capable of absorbing fluids that contact the fluid-contacting surface of the layer (i.e., the upper first surface of the fluid transport layer 30) and transporting or wicking those fluids away from the fluid contacting surface towards the absorptive reservoir layer 32. In a specific embodiment the fluid transport layer may be a textile material. By way of example only, such textile, materials may include knit textiles, woven textiles and non-woven textiles. Such textile materials may be formed from synthetic fibers, natural fibers or blends of synthetic and natural fibers as may be desired. The fluid transport layer 30 may be colored or printed. By way of example, the fluid transport layer may be dark colored or have a pattern printed in order to hide stains and fluids. The color and/or pattern requirements may be different for different applications such as hospital versus nursing care usage.

As noted, the fluid transport layer 30 includes an upper first surface having a first capillarity level and a lower second surface having a second capillarity level. In certain embodiments the capillarity of the upper first surface of the fluid transport layer 30 and the capillarity of the lower second surface of the fluid transport layer 30 may be substantially the same. In another embodiment the capillarity of the lower second surface of the fluid transport layer 30 which is disposed adjacent to the absorptive reservoir layer 32 may be greater than the capillarity level of the upper first surface of the fluid transport layer 30. In such an embodiment, the difference in capillarity levels between the upper and lower surfaces of the fluid transport layer 30 results in the lower surface of the fluid transport layer 30 exhibiting a greater affinity for aqueous fluids such as urine, blood, or wound exudates relative to the upper surface of the fluid transport layer 30. Thus, aqueous fluids that are absorbed by the fluid transport layer 30 will tend to be transported or pumped from the upper first surface towards the lower second surface. This active transportation or pumping aids in avoiding the accumulation of excess fluids at the surface of the top panel 12.

When the fluid transport layer 30 includes upper and lower surfaces having different capillarity levels, the difference between the two capillarity levels may be of any suitable magnitude. According to one exemplary embodiment, the capillarity level of the lower surface of the fluid transport layer 30 may be 101% or more of the capillarity of the upper surface of the fluid transport layer 30. Of course, greater differentials in capillarity levels may likewise be utilized if desired.

By way of example only, and not limitation, a fluid transport layer 30 having variable capillarity levels between the upper and lower surfaces may be selected from the group consisting of knit textiles, woven textiles, and non-woven textiles. Suitable knit textiles include, but are not limited to weft-knit textiles such as flat knit textiles and circular-knit textiles. FIG. 6 illustrates one exemplary knit fabric construction for a fluid transport layer 30 having surfaces with variable capillarity levels. In the illustrated embodiment, the fabric is a laid-in fabric having one or more structural yarns 34 providing the knit structure of the fabric and one or more effect yarns 36 tucked into the fabric structure. In order to provide two surfaces having different capillarity levels, the fluid transport layer 30 illustrated in FIG. 6 is a jersey knit fabric in which the effect yarns 36 are incorporated into the technical back of the fabric structure. Accordingly, the effect yarns 36 are disposed predominantly on the technical back of the fabric structure. Such a construction results in a fabric in which the technical face of the fabric is predominantly formed from the structural yarns 34 and the technical back presents a higher proportion of the effect yarns 36. Thus, when the structural yarns and the effect yarns 36 are formed from materials resulting in different contact angles, the capillarity levels of the technical face and the technical back will be different. By way of example, in an embodiment wherein the structural yarns 34 are polyamide yarns (e.g., nylon yarns) and the effect yarns 36 are polyester yarns the face of the fabric in which the more hydrophilic nylon yarns are present will present a higher capillarity than the face in which the polyester yarns predominate. In another embodiment, the structural yarns 34 are polyester. In another embodiment, all of the fibers in the fluid transport layer 30 are polyester. This serves to help the fluid transport layer 30 survive multiple commercial launderings and helps recycling when the layer 30 is ultimately disposed of. The yarns may be treated with chemistries to be hydrophilic.

An exemplary fluid transport layer 30 may further include yarns or fibers that provide the layer with the ability to be stretched and then returned to dimensions that are substantially the same as the original dimensions. For example, in addition to the components noted above, the structural yarns 34 may further include elastomeric fibers, or the fluid transport layer 32 may include elastomeric fibers or yarns that are disposed substantially parallel to the structural yarns 34. Alternatively, in addition to the effect yarns 36, the fabric may include elastomeric yarns or stretch yarns that have been tucked into the fabric structure. By way of example only, the elastomeric yarns may comprise materials such as SPANDEX®, LYCRA®, HYTREL® and the like, as will be well known to those in the art. In another embodiment, the fluid transport layer may contain yarn for low shear and low friction such as yarn from INVISTA®. The yarns in the fluid transport layer 30 and all other layers may be filament, staple, or a mixture of both.

In order to provide the differential capillarity levels described above, the fluid transport layer 30 may also comprise a material in which one surface is chemically or physically modified to yield a material having surfaces with different capillarity levels. For example, according to one exemplary practice, the fluid transport layer 30 may be a textile material such as those described above having an upper surface that has been chemically treated in order to lower the capillarity thereof. By way of example, the fluid transport layer 30 may be treated, for example, with a relatively hydrophobic fluorocarbon or silicone (i.e., a fluorocarbon or silicone that is more hydrophobic than the material forming the non-treated side of the textile material). In order to ensure that the use of such treatments does not produce a surface that is nonabsorbent, the chemical or physical modifications may be applied in such a manner as to produce a surface having a plurality of discreet discontinuities in the treatment. These discontinuities may provide a bypass pathway for fluid, thereby permitting the differential capillarity levels to transport or pump the fluid through the fluid transport layer 30 and into the absorptive reservoir layer 32.

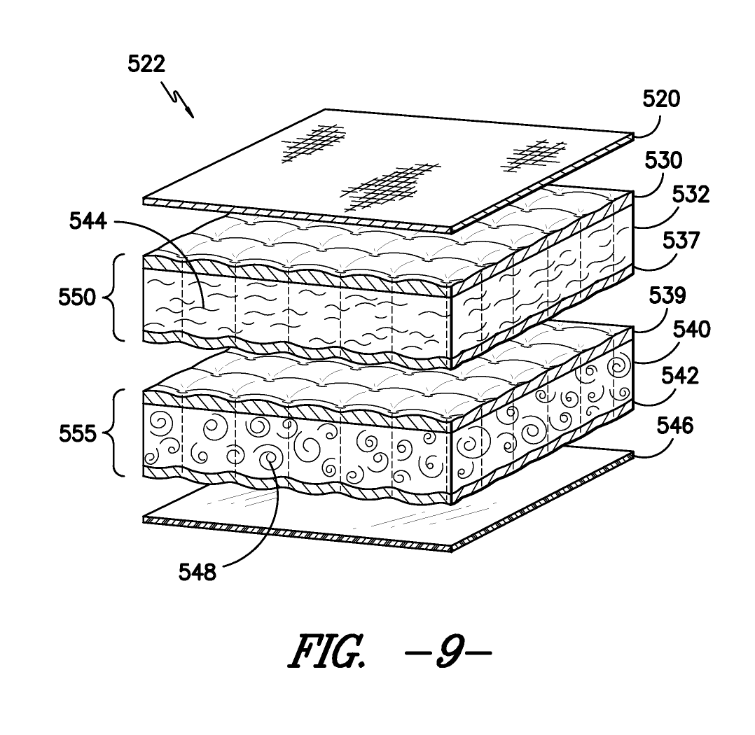

In accordance with one embodiment, the fluid transport layer 30 may be a pile fabric such as velour, velvet, or the like. FIG. 8 illustrates a typical pile fabric 27. As shown, such a pile fabric 27 includes a multiplicity of pile fiber elements 29 which extend outwardly away from a fibrous base 31. FIG. 8A illustrates the same pile fabric in which the pile fiber elements 29 have been randomized by physical treatments such as topical brushing, air impingement, water impingement or the like.

By way of example only, and not limitation, a pile fabric 27 may be formed using a sandwich method in which two fabrics are woven or knitted in face to face relation with the pile ends interlocking. A blade is used to slit through the pile ends to produce two separate pieces of fabric such that a multiplicity of pile fiber elements 29 project outwardly from the base 31. Such a technique is taught, for example, in U.S. Pat. No. 7,086,423 to Keller et al. the teachings of which are incorporated herein by reference. As will be appreciated, since the pile fiber elements 29 and the fibrous base 31 may be formed from different yarns, they may have different capillarity levels. The yarns also may be chemically treated to yield different capillarity levels at the pile fiber elements 29 and the fibrous base 31.

In accordance with one exemplary practice, the pile fiber elements 29 may define the upper surface of the fluid transport layer 30 and the fibrous base 31 may define the lower surface of the fluid transport layer 30. In this construction, when the upper surface is contacted with liquid, the liquid wicks along the pile fiber elements 29 and towards the fibrous base 31 where it is absorbed and spreads thereby leaving the upper surface relatively dry. The pile fiber elements 29 and the fibrous base 31 may be formed from similar or dissimilar materials. Such materials may include synthetic fibers, natural fibers or blends of synthetic and natural fibers. The pile fabric 27 may also include elastomeric or stretch yarns to improve stretch and recovery. Likewise, face finishing techniques such as disclosed in U.S. Pat. No. 6,866,911 to Demott et al. may be used to improve the softness or “hand” of the upper surface if desired.

By way of example only, the fibrous base 31 may be formed from a yarn characterized by a greater capillarity than the yarn making up the pile fiber elements 29. This differential in capillarity may be either inherent in the selection of materials or may be the result of chemical or physical treatment of the different yarn systems. Such a differential in capillarity facilitates the wicking of liquid along the pile fiber elements 29 and into the fibrous base 31.

The fluid transport layer 30 of the fluid management system 22 may exhibit any suitable absorptive capacity. For example, the fluid transport layer 30 can exhibit a fluid absorption (e.g., water absorption) of about 50% wt or more based on the weight of the fluid transport layer. In a specific embodiment, the fluid transport layer may exhibit a fluid absorption of about 100% wt or more, about 150% wt or more, about 200% wt or more, about 300% wt or more, or about 400% wt or more based on the weight of the fluid transport layer 30.

More information about a suitable fluid transport layer 30 may be found in US Patent Application Publication 2006/0127462 filed Feb. 3, 2006 (Canada et al.), which is herein incorporated by reference in its entirety.

The absorptive reservoir layer 32 may be any suitable material that is capable of retaining and absorbing fluids transported to the surface of the absorptive reservoir layer 32 by the fluid transport layer 30. By way of example only, the absorptive reservoir layer 32 may be selected from the group consisting of hydrophilic foams, textile materials including non-woven textile materials, alginates, superabsorbent polymers, gels (e.g., hydrogels), cellulosic fiber or batting, treated synthetic polymer fibers, hydro colloids, and combinations or mixtures thereof. The absorptive reservoir layer 32 may also include a combination of two or more discreet layers which can comprise any suitable absorptive materials. In addition, the absorptive reservoir layer 32 may include absorptive materials blended in combination with materials adapted to redistribute compressive forces as will be described further hereinafter.

The difference between the capillarity of the upper surface of the absorptive reservoir layer 32 and the lower surface of the fluid transport layer 30 may be of any suitable magnitude. For example, the capillarity of the upper surface of the absorptive reservoir layer 32 may be 105% or more of the capillarity of the adjacent lower surface of the fluid transport layer 30. In specific embodiments the capillarity of the upper surface of the absorptive reservoir layer 32 may be about 110% or more, about 115% or more, about 120% or more, or about 125% or more of the capillarity of the adjacent lower surface of the fluid transport layer 30.

The absorptive reservoir layer 32 may exhibit any suitable absorptive capacity. For example, the absorptive reservoir layer 32 may exhibit an absorption of about 100% wt or more based on the weight of the absorptive reservoir layer 32. In specific embodiments, the absorptive reservoir layer 32 may exhibit a fluid absorption level of about 200% wt or more, about 300% wt or more, about 400% wt or more, about 500% wt or more, about 600% wt or more, about 700% wt or more, about 800% wt or more, about 900% wt or more, or about 1,000% wt or more based on the weight of the absorptive reservoir layer 32. In this regard, absorptive capacity of the absorptive reservoir layer 32 may be measured by any suitable means. For example, the absorptive capacity may be measured by immersing a known weight of the absorptive reservoir layer in phosphate-buffered saline containing about 0.9% wt sodium chloride at 37° C. until saturation and then weighing the saturated product.

As noted previously and depicted in FIG. 2, the composite forming the top panel 12 or seating pad 17 of the fluid management system 22 further includes a pressure redistribution layer 40 disposed at a position below the absorptive reservoir layer 32. The pressure redistribution layer is adapted to compress in response to applied pressure such as from the user and to then recover substantially to its original configuration. The pressure redistribution layer 40 may be formed of any suitable material which exhibits compression and recovery in a wet state. By way of example only, the pressure redistribution layer may comprise either synthetic materials or a blend of synthetic and natural materials. The pressure redistribution layer 40 may also comprise mechanical structures including air or fluid bladder systems and the like that can be filled and manipulated by a user as well as passive air or fluid filled systems to yield a desired degree of firmness and resiliency.

The pressure redistribution layer 40 may be either a substantially unitary structure or may be formed from a mass of discrete elements that are contained within a given zone. Suitable exemplary materials may include hydrophobic foams, such as polyurethane foam, memory foam, TPU foam, olefin foam, silicone foam, rubber foam, and the like. Other suitable materials may include non-woven textiles including high-loft non-woven textiles, spacer fabrics, and pile or cut-pile fabrics. Accumulations of discrete resilient elements such as resilient particulate balls, foam particles or chunks, or discrete compressible fiber elements as well as any combinations or mixtures of such materials may also be used. By way of example only, in accordance with one exemplary embodiment the pressure redistribution layer 40 my comprise an accumulated mass of so called “fiberfill” or cluster fibers of polyester or other suitable material as may be desired. By way of example, suitable compressible fiber ball structures are disclosed in U.S. Pat. No. 4,794,038 to Marcus, the contents of which are hereby incorporated by reference in their entirety. The pressure redistribution layer may be used to help prevent pressure ulcers or may be used to help treat pressure ulcers.

As noted, the purpose of the pressure redistribution layer 40 is to exhibit compression and recovery characteristics in response to applied localized loading from a user or other source. In this regard, the pressure redistribution layer 40 will exhibit recovery levels following loading of at least about 50% or more in a wet state. In specific embodiments, the pressure redistribution layer 40 may exhibit compressive recovery of about 60% or more, about 70% or more, about 80% or more, or about 90% or more in the wet state.

It is to be understood that the pressure redistribution layer 40 may be comprised of either a single layer or multiple layers if desired. Likewise, the pressure redistribution layer 40 may define either a discrete independent layer or may be combined with the absorptive reservoir layer 32 to yield both absorption and pressure redistribution in manners as will be described further hereinafter.

As previously noted and illustrated in FIG. 2, the composite forming the top panel 12 of the mattress cover or seating pad 17 may include an optional backing layer 42. The backing layer 42 may provide a base surface for containment of overlying cluster fibers or other particulate matter used within overlying layers. In this regard, the backing layer 42 may be formed from any suitable containment material as may be desired. Exemplary suitable materials include textiles including woven, non-woven and knit structures, films, and the like. The backing layer 42 may be formed from a substantially hydrophobic material such as polyester or the like so as to substantially avoid moisture retention. The backing material may also be formed of natural materials which are treated to provide enhanced levels of hydrophobicity. The backing layer 42 may also be treated with a repel/release chemistry and may be moisture impermeable.

As previously noted and illustrated in FIG. 2, the composite structure forming the top panel 12 or seating pad 17 may have a quilted construction including an arrangement of quilting yarns 44 extending in a quilting pattern between the backing layer 42 and the surface of the fluid transport layer 30. Thus, the quilting yarns 44 will substantially perforate the backing layer, pressure redistribution layer 40, absorptive reservoir layer 32, and fluid transport layer 30. The quilting yarns 44 will preferably be arranged at a controlled spacing across the width of the top panel 12 to provide a suitable containment function of the various layers without applying undue compression to the overall composite. According to specific embodiments, the spacing between the quilting yarns may be about 2 inches or greater, about 3 inches or greater, about 4 inches or greater, about 5 inches or greater, about 6 inches or greater, about 7 inches or greater, about 8 inches or greater, about 9 inches or greater, or about 10 inches or greater. In this regard, it will be understood that the quilting yarns provide a binding function which acts to hold the layers of the composite together in a substantially cohesive manner. The quilting yarns 44 may be formed from any suitable synthetic or natural material having a sufficient tenacity to provide the requisite binding function. The quilting yarns 44 may be either hydrophobic or hydrophilic. However, hydrophobic yarns such as polyester yarns or the like may be particularly suitable for many applications. While the use of quilting structures may be beneficial for many applications, it also contemplated that adhesive layers (not shown) may be interposed in binding relation between any of the various structural layers. Such adhesive layers may be discontinuous across the interface to facilitate fluid transfer between layers. Other techniques for attachment including ultrasonic welding, lasers, heat sealing and the like may also be used if desired.

As depicted in FIG. 2, the composite forming the top panel 12 of the fluid management system 22 or seating pad 17 may further include an optional strike-through layer disposed at a position below the quilted layers. That is, the quilting yarns 44 do not pass through the strike-through layer 46. Thus, fluid is blocked from traveling along the pathways established by the quilting yarns 44 to the underside of the top panel 12 or seating pad 17. The strikethrough layer 46 may be formed from any suitable material including a textile such as a woven, non-woven, or knit textile. The strikethrough layer 46 may also be formed from a suitably pliable film. Textile materials of substantially hydrophobic character may be particularly desirable. In practice, the strikethrough layer 46 may be applied across the backing layer 42 and held in place by a perimeter stitch line (not shown) which extends about the perimeter of the interface with the backing layer. In this manner, the surface area of the strikethrough layer 46 remains substantially unperforated thereby substantially obstructing capillary migration of the fluid across the strikethrough layer 46.

FIG. 3 depicts an alternative embodiment wherein elements corresponding to those previously described are designated with like reference numerals within a 100 series. In the embodiment of FIG. 3, the materials providing the absorptive reservoir function and the materials providing the pressure redistribution function may be blended into one or more common layers. Accordingly, in such a construction the blended layer is both an absorptive reservoir layer and a pressure redistribution layer. In the embodiment of a fluid management system 122 illustrated in FIG. 3, a skin contacting layer 120 as previously described is disposed in unconnected opposing relation to a quilted composite forming a top panel 112 of a mattress cover or seating pad as previously described. The composite forming the top panel 112 or seating pad includes a fluid transport layer 130 disposed above a dual function reservoir and pressure redistribution layer 150 which contains both absorptive reservoir elements and pressure redistribution elements as previously described. In specific embodiments, the reservoir and pressure redistribution layer 150 may comprise a blend of cellulosic or foam elements dispersed in combination with material such as hydrophobic foams, polyester cluster fibers or the like to form a blend which provides features of both fluid absorption and pressure redistribution. The ratios of fluid absorption elements to pressure redistribution elements may be selected as desired. In this regard, the blend may comprise between about 10% wt to about 90% wt of the fluid absorptive component in combination with about 10 wt. % to about 90 wt. % of the pressure redistribution component. According to specific examples, the reservoir and pressure redistribution layer 150 may comprise about 30% wt to about 70% wt of the absorptive reservoir component in combination with about 70% wt to about 30% wt of the pressure redistribution component. According to another specific embodiment, the dual function layer 150 may comprise about 40% wt to about 60% wt of the absorptive reservoir component and about 60% wt to about 60% wt of the pressure redistribution component. According to yet another specific construction, the dual function layer 150 may comprise about 50% wt of the absorptive reservoir component and about 50% wt of the pressure redistribution component.

In the exemplary arrangement illustrated in FIG. 3, the composite forming the top panel 112 or seating pad further includes a backing layer 142 as previously described for purposes of containing the materials forming the dual function layer 150. Quilting yarns 144 as previously described extend in binding relation from the backing layer 142 to the fluid transport layer 130. A strikethrough layer 146 as previously described may also be provided. As noted previously, such a strikethrough layer 146 may be secured about the perimeter of the interface with the backing layer 142 to avoid establishing fluid transfer channels at the interior of the strikethrough layer 146.

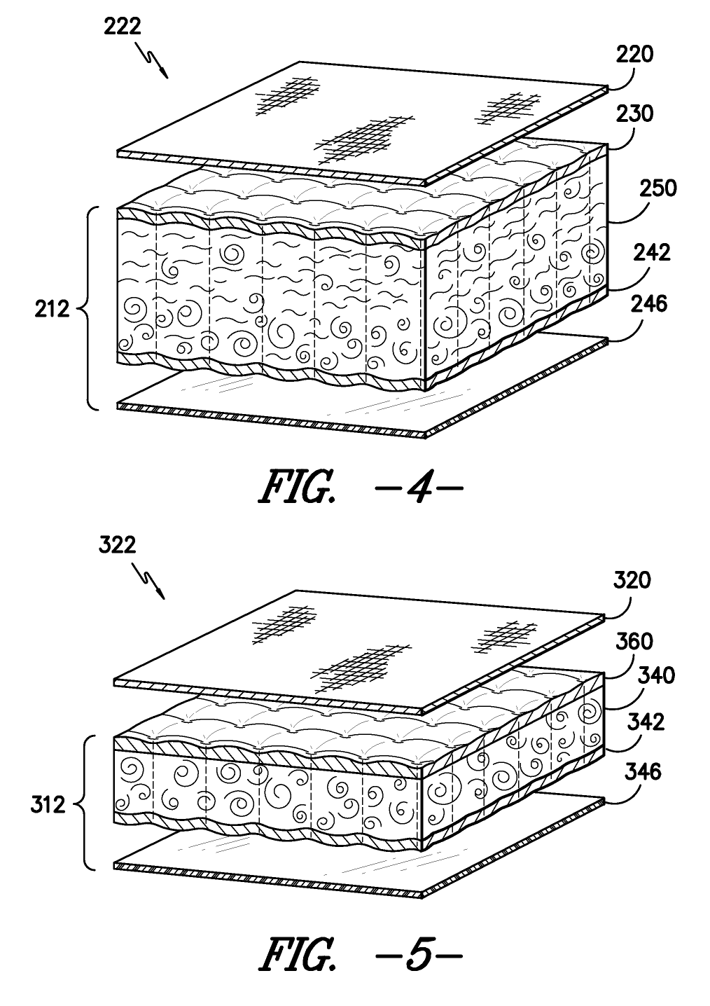

FIG. 4 depicts a variant of the construction illustrated in FIG. 3 wherein elements corresponding to those previously described are designated with like reference numerals within a 200 series. In the embodiment illustrated in FIG. 4, an optional skin contacting layer 220 is illustrated in overlying relation to a composite structure forming a top panel 212 or seating pad in a fluid management system 222. The construction of the composite forming the top panel 212 is substantially the same as described in relation to FIGS. 2 and 3. However, the specific embodiment illustrated in FIG. 4 incorporates a dual function reservoir and pressure redistribution layer 250 providing a variable distribution of absorptive reservoir components such as cellulosic materials, hydrophilic foams, or the like, and pressure redistribution components such as hydrophobic foams, polyester cluster fibers, or the like, as previously described. If desired, synthetic fibers such as polyester or other constituents within the dual function reservoir and pressure redistribution layer 250 may be treated to increase hydrophilicity.

In the embodiment illustrated in FIG. 4, the absorptive reservoir components are disposed at a substantially higher concentration adjacent the upper surface of the reservoir and pressure redistribution layer 250 while the pressure redistribution components have a substantially higher concentration adjacent the lower surface of the reservoir and pressure redistribution layer 250. Thus, the absorptive reservoir components are arranged in a gradient of decreasing concentration from the upper surface to the lower surface with the pressure redistribution components being arranged in a substantially inverse gradient with higher concentrations at the lower surface and reduced concentrations at the upper surface. This construction provides the reservoir and pressure redistribution layer 250 with the desired high capillarity level to draw fluid away from the fluid transport layer 230 while at the same time, providing substantial pressure redistribution character. The fluid transport layer 230 may be treated to additional processes such as sanding if the optional skin contact layer is not in the system.

By way of example only, and not limitation, one technique for forming such a gradient structure is to use a commercially available piece of equipment such as the (K-12 HIGH LOFT RANDOM CARD) from Fehrer AG having a place of business in Linz, Austria. Such equipment operates by applying a blend of materials to a rotating cylinder which slings the blended material towards a collection belt. The spinning rotation of the cylinder throws the heavier constituents of the blend a further distance than the lighter constituents. As a result, a mat of the constituents may be built up on the collection belt with a greater concentration of the heavier components near the top of the matted construction. In general, the larger the difference in density between the constituents, the more pronounced the gradient in concentration will be. Of course, virtually any other method as may be desired to build a dual function layer with a higher concentration of absorptive reservoir components adjacent an upper side may likewise be used if desired.

In accordance with one specific embodiment, a gradient structure of fluid absorptive elements in combination with pressure redistribution elements formed by a K-12 process or the like may be oriented within the composite forming the top panel 212 or seating pad with the surface formed predominantly from the pressure redistribution elements of generally lower hydrophilicity facing downwardly towards the backing layer 242 and the surface with the higher percentage of fluid absorptive elements with generally higher hydrophilicity facing upwardly towards the fluid transport layer 230. In such a structure, the upper surface will act to draw fluid away from the overlying fluid transport layer 230 for retention within the reservoir and pressure redistribution layer 250.

In accordance with another specific embodiment, a gradient structure of fluid absorptive elements in combination with pressure redistribution elements formed by a K-12 process or the like may be oriented within the composite forming the top panel 212 or seating pad with the surface formed predominantly from the pressure redistribution elements of generally lower hydrophilicity facing upwardly in adjacent relation to the fluid transport layer and with the surface with the higher percentage of fluid absorptive elements with generally higher hydrophilicity facing downwardly towards the backing 242. In such a structure, the upper surface formed predominantly from the pressure redistribution elements may act to draw fluid away from the overlying fluid transport layer 230 so long as it has a higher capillarity than the overlying fluid transport layer 230. Fluid may then be passed towards the surface with the higher percentage of fluid absorptive elements for retention.

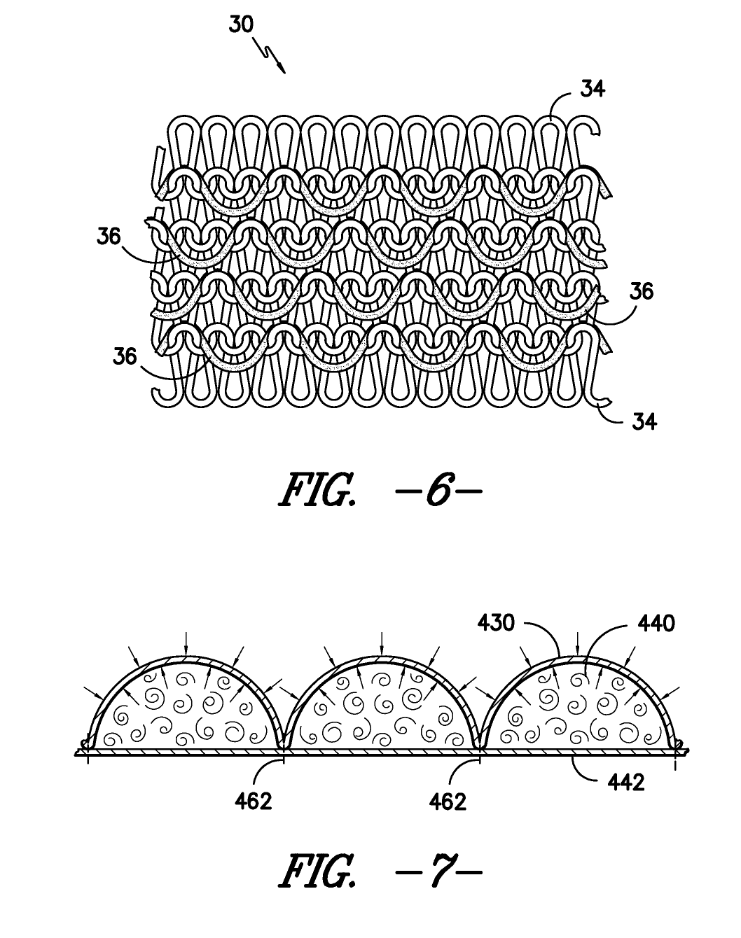

FIG. 5 depicts an alternative construction wherein elements previously described are designated by like reference numerals within a 300 series. As can be seen, the fluid management system 322 illustrated in FIG. 5 is substantially similar to that illustrated in FIG. 2 but with the elimination of the discrete absorptive layer. Rather, both fluid transport and absorptive reservoir functions are provided by a common dual function transport and reservoir layer 360 disposed above a pressure redistribution layer 340 of cluster fibers or other materials as previously described. In this arrangement, the transport and reservoir layer 360 may be characterized by a capillarity gradient between its upper first surface and lower second surface. That is, the capillarity at the upper surface which initially contacts the expelled fluid is lower than the capillarity of the lower surface opposing the pressure redistribution layer 340. This arrangement permits the lower portion of the transport and reservoir layer 360 to draw fluid away from the upper portion of the transport and reservoir layer 360 and to substantially retain such fluid above the pressure redistribution layer 340.

A transport and reservoir layer 360 having a variable capillarity gradient through its thickness may be formed in a number of ways. In accordance with one exemplary practice, such a transport and reservoir layer 360 may have a structure substantially as described in relation to FIG. 6, in which an effect yarn 36 of greater hydrophilicity is disposed predominantly across a lower face of the transport and reservoir layer 360 while structural yarns 34 of lower hydrophilicity form the predominant portion of the upper face of the transport and reservoir layer 360. As previously described, such an arrangement causes the lower face containing the predominant portion of the effect yarns to have a higher capillarity than the upper surface, thereby drawing fluid away from the upper surface for retention at the lower surface. A relatively lower capillarity level at the upper surface of the pressure redistribution layer 340 will act to restrict further migration into the pressure redistribution layer 340.

In accordance with another exemplary embodiment, a transport and reservoir layer 360 having a gradient of capillarity levels may be formed by a procedure in which fibers or other elements with different hydrophilicity levels are formed into a matted structure using an apparatus such as the “K-12” apparatus. As previously noted, such an apparatus achieves a gradient distribution of different fiber types by applying a desired blend of fibers to a rotating cylinder which slings the blended fibers towards a collection belt. The spinning rotation of the cylinder slings the heavier fibers a further distance along the collection belt than it slings the lighter fibers. As a result, a mat of fibers collected on the collection belt will have a greater concentration of the lighter fibers adjacent to the collection belt and a greater concentration of the heavier fibers further away from the collection belt. Accordingly, applying this practice the transport and reservoir layer 360 may be formed from two or more different fiber types with different hydrophilicity levels and densities. The fiber type with the higher density will be disposed predominantly at one side of the formed fibrous mat while the fiber type with the lower density will be disposed predominantly at the other side of the fibrous mat. In accordance with one specific embodiment, the fibrous mat may then be oriented within the composite forming the top panel 312 or seating pad with the surface formed predominantly from the fiber type of higher hydrophilicity facing downwardly towards the pressure redistribution layer 340 and the surface with the higher percentage of fibers with reduced hydrophilicity facing upwardly away from the pressure redistribution layer 340. In such a structure, the lower surface will act to draw fluid away from the upper surface and to retain such fluid at a position above the pressure redistribution layer 340.

The absorptive capacity for the transport and reservoir layer 360 may be readily adjusted by adjusting the percentage of fibers with higher and lower hydrophilicity levels and absorptive capacities. In this regard, by selecting a high percentage of fibers with substantial absorptive capacity, the overall absorptive capacity of the fluid transport and reservoir layer 360 may be increased while utilizing a lower percentage of fibers with significant absorptive capacity will reduce the overall absorptive capacity.

As noted previously, in general, using fiber types of substantially different densities will yield a steeper gradient within the transport and reservoir layer 360 formed by a “K-12” machine or the like. Thus, by selecting fiber types of substantially different densities, a steep concentration gradient may be achieved between the upper and lower surfaces of the transport and reservoir layer 360. Conversely, by selection of fiber types with substantially similar densities a relatively shallow gradient of fiber concentrations may be established. Accordingly, the character of the transport and reservoir layer 360 may be adjusted to provide both a desired fluid pumping action in which fluid is drawn away from the upper surface as well as a controlled absorptive capacity in which the fluid is held in a zone adjacent to the lower surface.

In accordance with another exemplary embodiment, a transport and reservoir layer 360 having a gradient of capillarity levels may be a pile fabric as previously described in relation to FIGS. 8 and 8A. In such a construction, the pile fiber elements will provide the transport function while the base 31 defines a reservoir for fluid retention.

As noted previously, it is contemplated that incorporating materials of elastomeric character into one or more layers within a composite structure forming the top panel of the mattress cover may be beneficial in some environments of use. In this regard, elastomeric character may be built into anyone of the fluid transport layer, the absorptive reservoir layer, the pressure redistribution layer, the optional backing layer, or any combination thereof. According to one specific embodiment, the fluid transport layer may be a knit or woven fabric with a high stretch component provided by yarn components such as LYCRA®, SPANDEX®, HYTREL®, or the like.

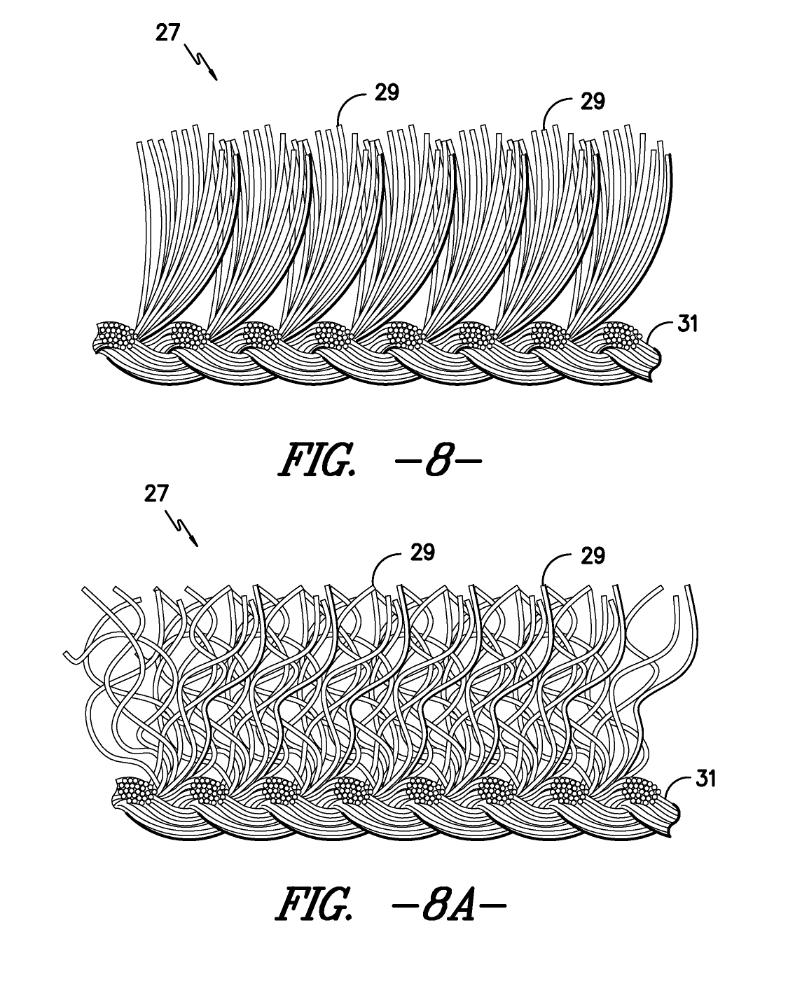

By way of example only, FIG. 7 illustrates an exemplary arraignment in which an elastomeric layer 430 such as a fluid transport layer as previously described is disposed in overlying relation to a pressure redistribution material 440 such as polyester cluster fibers, hydrophobic foam, or the like. If desired, one or more intermediate layers such as a fluid reservoir layer (not shown) may be disposed between the elastomeric layer 430 and the pressure distribution material 440. In the illustrated configuration, a backing layer of elastic or inelastic character provides containment for the pressure redistribution material 440 and serves as a base for the application of tie-down points 462 between the elastomeric layer 430 and the backing layer 442. The tie-down point 462 may be quilting lines or other connective techniques which yield a zero length or finite length tethering arrangement between the overlying elastomeric material and the backing layer 442. The tie-down points 462 are applied and maintained such that the elastomeric layer 430 or other overlying elastomeric layer as may be used is held in a continuously tensioned state over the pressure redistribution material 40 or other intermediate media which may be present between the elastomeric material and the underlying backing.

As shown by the force arrows in FIG. 7, by securing the elastomeric layer 430 or other elastomeric structure as may be utilized in a tensioned state over a resilient filling material a substantially stable highly resilient structure is established. Specifically, as the overlying elastomeric material presses inwardly, the underlying fill material applies continuous outward resistance to establish equilibrium. As external pressure is applied, both the elastomeric covering and the underlying fill material will resist deformation and will continuously seek to reestablish a force-neutral condition. Thus, recovery following the application of a compressive force is facilitated by both the elastomeric covering layer as well as the underlying resilient fill material.

In accordance with a particular embodiment, the overlying elastomeric layer may be a fabric such as a circular knit, circular knit with lay-in, double knit, warp knit, or the like. In accordance with a particular embodiment such a fabric may have greater than about 15% strain in tension prior to failure, greater than about 40% strain in tension prior to failure, greater than about 65% strain in tension prior to failure, greater than about 90% strain in tension prior to failure, or greater than about 120% strain in tension prior to failure. Such materials may have a modulus of between about 0.05 and about 4 pounds force per inch width per strain (where strain is inch per inch). The initial strain at rest following formation may be between about 0.1% and about 25%. The elastomeric covering material may either be free from the underlying fill material or may be adhered thereto. In accordance with one particular embodiment the overlying elastomeric material may be a knit fabric comprising about 5% wt or greater SPANDEX® yarn, about 10 wt % or greater SPANDEX® yarn, or about 20% wt or greater SPANDEX® yarn. The backing layer 442 may be a similar or dissimilar construction to the overlying elastomeric layer.

In accordance with one particular embodiment, the backing layer may be a woven, knit or non-woven fabric having about 0% wt to about 20% wt SPANDEX® or other elastomeric fiber constituents. In this regard, a backing layer 442 of substantially inelastic character may be desirable in many applications to promote stability while simultaneously reducing costs. The spacing of the tie-down points 462 may be adjusted as desired to yield desired resiliency levels. In accordance with one exemplary arrangement, the tie-down points may be formed by quilting spaced at distances of about 1 inches to about 12 inches, and more preferably at spacing intervals of about 2 inches to about 7 inches.

In accordance with one exemplary embodiment, the elastomeric layer 430 may be a textile layer with a variable capillarity level between surfaces as previously described.

Rather, both fluid transport and absorptive reservoir functions are provided by a common dual function transport and reservoir layer 360 disposed above a pressure redistribution layer 340 of cluster fibers or other materials as previously described

FIG. 9 depicts an alternative construction wherein elements previously described are designated by like reference numerals within a 500 series. As can be seen, the fluid management system 522 illustrated in FIG. 9 is substantially similar to that illustrated in FIG. 2 but with the separation of the fluid transport section 550 and the pressure redistribution section 555. One advantage of having the two sections separate (not permanently adhered) is that the fluid transport section 550 may be taken off to be laundered easily while leaving the pressure redistribution section 555 on the mattress, chair, or other article. The fluid transport section 550 may be replaced while leaving the pressure redistribution section 555 on the mattress, chair, or other article. The skin contact layer 520 may be any suitable material as previously described.

The fluid transport section 550 contains the fluid transport layer 530, previously described in relation to FIG. 2. The fluid transport section 550 may also contain an optional absorptive reservoir layer 532 and/or an optional backing layer 537. The optional backing layer may be any suitable backing layer described in FIG. 2 as part 42. The layers of the fluid transport section 550 may be discrete layers or the fluid transport section 550 may contain one layer that combines the functionality of two or more of the layer 530, 532, 537. For example, a layer may be used like layer 150 from FIG. 3 combining the functionality of the fluid transport layer 530 and the absorptive layer 532 into one layer. Such as layer may be a two or three layer multi-layer fabric (such as a double knit and three-dimensional woven fabrics), such as described in U.S. Pat. No. 7,473,659 (Sytz), which is incorporated herein by reference in its entirety. The fluid transport section 550 may also have optional quilting 544 which may have the same materials and physical characteristics as the quilting shown in FIG. 2. The quilting 544 may pass through the entire fluid transport section 550 or any combination of layers within the fluid transport section 550 such as the fluid transport layer 530 and the backing layer 537 or the fluid transport layer 530 and the absorptive reservoir layer 532.

The pressure redistribution section 555 contains the pressure redistribution layer 540, previously described in relation to FIG. 2. The pressure redistribution section 555 may also contain an optional top layer 539 and/or an optional backing layer 542. The optional top layer 539 and the optional backing layer 542 may be any suitable backing layer 42 described previously in FIG. 2. In another embodiment, the optional top layer 539 may be water and/or vapor permeable and cleanable. This may be accomplished, for example, by adding a fluorochemical treatment to the outer surface of the top layer 539. The layers of the pressure redistribution section 555 may be discrete layers or the pressure redistribution section 555 may contain one layer that combines the functionality of two or more of the layers 539, 540, 542. The pressure redistribution section 555 may also have optional quilting 548 which may have the same materials and physical characteristics as the quilting (44) shown in FIG. 2. The quilting 548 may pass through the entire pressure redistribution section 555 or any combination of layers within the pressure redistribution section 555 such as the pressure redistribution layer 540 and the top layer 539 or the pressure redistribution layer 540 and the backing layer 542. There may be additional layer in the pressure redistribution layer 555 over the optional top layer 539 that is not quilted on, but may be attached along the edges. This additional layer may be wipe cleanable and may have chemistries such as fluorochemicals applied to at least one surface of it.

The fluid transport section 550 and the pressure redistribution section 555 may be separate or connected in a suitable manner. Some attaching means include, but are not limited to, zippers, VELCRO®, snap buttons, buttons, skirt, or straps. Skirt and strap mean that a piece of fabric (such as an elastic band) is sewn on the edge of the fluid transport section 550 or the pressure redistribution section 555 in order to secure them around the mattress, chair, or other article.

In addition to the physical construction features outlined above, it is also contemplated that any of the layers or combination of the layers within the fluid management systems described may be subjected to anti-microbial or odor control treatments. By way of example only, such treatments may include the application of Urease inhibitors, silver containing antimicrobials, activated carbon, or the like. In addition, it is contemplated that any of the layers may be provided with a wetness indicator in the form of an activated colorant or the like to indicate the condition of the layer.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A fluid management system comprising:
at least one absorptive reservoir layer having an upper surface and a lower surface;
at least one fluid transport layer disposed on the upper surface of the absorptive reservoir in an overlying relation, wherein the upper surface of the absorptive layer has a greater capillarity than the adjacent surface of the fluid transport layer such that fluid is drawn away from the fluid transport layer; and,
at least one pressure distribution layer disposed on the lower surface of the absorptive reservoir in an underlying relation, wherein the pressure redistribution layer comprises a material that at least partially recovers following compression.

2. The fluid management system for mattresses comprising the fluid management system of claim 1 and a mattress, wherein the fluid management system overlays the mattress.

3. The fluid management system of claim 2, wherein each layer of the fluid management system completely covers the mattress.

4. The fluid management system for seating comprising the fluid management system of claim 1 and a seating structure, wherein the fluid management system overlays at least a portion of the seating structure.

5. The fluid management system of claim 4, wherein each layer of the fluid management system completely covers the seating structure.

6. The fluid management system of claim 1, further comprising a skin contact layer disposed on the fluid transport layer, on the side opposite the absorptive reservoir layer.

7. The fluid management system of claim 1, wherein the at least one absorptive reservoir, the at least one fluid transport layer, and the at least one pressure distribution layer are quilted together.

8. The fluid management system of claim 1, wherein the at least one fluid transport layer comprises a pile fabric.

9. The fluid management system of claim 1, wherein the system further comprises a wipe cleanable layer.

10. The fluid management system of claim 1, wherein the absorptive reservoir layer is selected from the group consisting of hydrophilic foams, textile materials, alginates, superabsorbent polymers, gels, cellulosic fiber, cellulosic batting, treated synthetic polymer fibers, hydro colloids, and mixtures thereof.

11. The fluid management system of claim 1, wherein the pressure distribution layer comprises cluster fibers.

12. The fluid management system of claim 1, wherein the at least one absorptive reservoir layer and the at least one fluid transport layer are combined into one dual function transport and reservoir layer having an upper and lower surface, wherein the upper surface has a greater capillarity than the lower surface.

13. The fluid management system of claim 1, wherein the at least one absorptive reservoir layer and the at least one pressure redistribution layer are combined into one dual function layer having an upper and lower surface, wherein the upper surface has a greater capillarity than the lower surface, wherein the dual function layer comprises both absorptive reservoir components and pressure redistribution components, wherein the dual layer comprises a gradient where the absorptive reservoir components are at the highest concentrations at the dual function layer upper surface and decrease in a gradient to the lower surface and the pressure redistribution components are at the highest concentrations at the dual function layer lower surface and decrease in a gradient to the upper surface.

14. A fluid management system comprising:
at least one absorptive reservoir layer having an upper surface and a lower surface;
at least one fluid transport layer disposed on the upper surface of the absorptive reservoir in an overlying relation, wherein the upper surface of the absorptive layer has a greater capillarity than the adjacent surface of the fluid transport layer such that fluid is drawn away from the fluid transport layer; and,
at least one pressure distribution layer disposed on the lower surface of the absorptive reservoir in an underlying relation, wherein the pressure redistribution layer comprises a material that at least partially recovers following compression;
wherein the at least one fluid transport layer and the at least one pressure distribution layer are not permanently adhered to one another.

15. The fluid management system for mattresses comprising the fluid management system of claim 14 and a mattress, wherein the fluid management system overlays the mattress.

16. The fluid management system of claim 15, wherein each layer of the fluid management system completely covers the mattress.

17. The fluid management system for seating comprising the fluid management system of claim 14 and a seating structure, wherein the fluid management system overlays at least a portion of the seating structure.

18. The fluid management system of claim 14, wherein the at least one fluid transport layer comprises a pile fabric.

19. The fluid management system of claim 14, wherein the system further comprises a wipe cleanable layer.

20. The fluid management system of claim 14, wherein the at least one absorptive reservoir layer and the at least one fluid transport layer are combined into one dual function transport and reservoir layer having an upper and lower surface, wherein the upper surface has a greater capillarity than the lower surface.
